(19) United States
(12) Patent Application Publication
(10) Pub. No.: US 2004/0086526 A1
(43) Pub. Date: May 6, 2004

(54) Process for protecting the skin against aging

(76) Inventors: Louis Danoux, Saulxures les Nancy (FR); Olga Freis, Seichamps (FR); Gilles Pauly, Nancy (FR)

Correspondence Address: COGNIS CORPORATION, PATENT DEPARTMENT, 300 BROOKSIDE AVENUE, AMBLER, PA, 19002, US

(21) Appl. No.: 10/620,786

(22) Filed: Jul. 16, 2003

(30) Foreign Application Priority Data
Jul. 17, 2002 (EP) 02291799.1

(51) Int. Cl. A61K 31/685; A61K 35/84; A61K 38/26

(52) U.S. Cl. 424/195.15; 514/46; 514/78; 514/8; 514/474; 424/195.16

(57) ABSTRACT

The invention relates to a process for the production of preparations for stimulating human skin cells and for protection against aging of the skin and the harmful effects of environmental toxins and UV radiation, characterized in that the preparations contain at least one substance which increases the synthesis of energy donors of the mitochondrial respiratory chain and, at the same time, lowers the level of reactive oxygen species (ROS) in the cell metabolism. The invention also relates to the use of cosmetic and/or pharmaceutical preparations containing at least one substance which increases the synthesis of energy donors of the mitochondrial respiratory chain and, at the same time, lowers the level of reactive oxygen species (ROS) in the cell metabolism for stimulating human skin cells and for protection against aging of the skin and against the harmful effects of environmental toxins and UV radiation.

### BACKGROUND OF THE INVENTION

Mitochondria are intracellular organelles which are the most important elements for the energy production of the cells. The contain a complete supply of enzymes for the degradation of fatty acids, the citric acid cycle, oxidative phosphorylation and the respiratory chain and are chiefly responsible for the energy supply and the oxidation of various molecules in the final stage of the aerobic metabolism of cells.

Accordingly, these organelles are a highly sensitive indicator for stress factors, such as toxic environmental poisons, UV-A or UV-B radiation and also natural aging. Stress reduces the capacity of the mitochondria to produce energy. Mitochondria thus damaged release an increased quantity of so-called reactive oxygen species (ROS) and other factors, such as cytochrome C which initiates cell death in the form of apoptosis. These ROS are basically released as secondary products of electron transport in the mitochondrial respiratory chain. For example, hydrogen peroxide is formed during the reaction of succinate, the most effective substrate of the respiratory chain. The ROS are in turn captured by a protection mechanism of the cells in the form of enzymes which bind or react free oxygen radicals, because they would otherwise damage cellular macromolecules, such as proteins, lipids and nucleic acids.

However, the level of ROS in the cells increases with increasing age because the activity of the oxygen-radical-binding enzymes decreases as does the effectiveness of electron transport in the mitochondrial respiratory chain.

It is known that UV-A- and UV-B radiation, by damaging the mitochondria, also produce an increase in the ROS which then contributes to damage to cellular macromolecules.

Accordingly, International patent application WO 98/51291 discloses the use of antioxidants for protecting cells and tissue against ROS which were increasingly released by ischemic processes of mitochondria. The use of L-ergothionine for protecting mitochondria against oxidative damage by UV radiation and environmental toxins has also been described (WO 98/36748 and U.S. Pat. No. 6,103,746).

Another way of slowing down the aging process is to increase the activity of the mitochondria. Thus, it was shown in Japanese patent application JP 08333270 that the use of extracts of tapra fruit increases the effectiveness of the mitochondria. Unfortunately, the release of ROS is also increased.

Accordingly, there was still a need to find mechanisms which would reduce cell aging or damage by environmental toxins or UV radiation. This was the problem addressed by the invention.

### SUMMARY OF THE INVENTION

This invention relates generally to cosmetic and pharmaceutical preparations and, more particularly, to a process for protecting human skin against aging and against the harmful effects of environmental toxins and UV radiation. The invention also relates to the use of cosmetic or pharmaceutical preparations containing at least one substance which increases the synthesis of energy donors of the mitochondrial respiratory chain and, at the same time, reduces the level of reactive oxygen species (ROS) in the cell metabolism for stimulating human skin cells and for protection against aging of the skin, toxic environmental influences and UV radiation.

The present invention relates to processes for the production of preparations for stimulating human skin cells, preparations for protecting human skin against aging, preparations for protection against harmful effects and aging of the skin by UV radiation and preparations for protecting the human skin against toxic environmental influences, characterized in that the preparations contain at least one substance which increases the synthesis of energy donors of the mitochondrial respiratory chain and, at the same time, lowers the level of reactive oxygen species (ROS) in the cell metabolism.

It has surprisingly been found that the human skin can be more effectively protected against aging and against damage by environmental poisons and UV radiation by a process in which, on the one hand, the mitochondrial function is stimulated but, on the other hand, the level of reactive oxygen species released as a result is not increased. The increase in the synthesis of energy donors of the mitochondrial respiratory chain, such as adenosine triphosphate (ATP) and/or creatine phosphate for example, supports cell-physiological mechanisms which, on the one hand, preventively protect the skin against damage and which, on the other hand, promote the repair mechanism for already damaged skin.

During the natural aging process, the energy provided by the mitochondria steadily decreases while the level of reactive oxygen species that are not detoxified in the cell steadily increases. It is also known that UV radiation can cause damage to the respiratory chain which also leads to a reduced potential for energy production and, at the same time, to an increased ROS level.

Accordingly, one objective was to increase the energy production capacity of aging skin. In the cell metabolism, the level of ROS released is increased by the induction of the mitochondrial function. In the process according to the invention, however, the quantity of ROS from the mitochondrial respiratory chain is intended to be reduced despite the increased synthesis of energy donors of the respiratory chain.

The present invention also relates to the use of cosmetic and/or pharmaceutical preparations containing at least one substance which increases the synthesis of energy donors of the mitochondrial respiratory chain and, at the same time, lowers the level of reactive oxygen species (ROS) in the cell metabolism for stimulating human skin cells and/or for protecting human skin against aging and/or for protecting the skin against harmful effects and aging of the skin by UV radiation and/or for protecting human skin against toxic environmental influences.

The possible energy donors of the mitochondrial respiratory chain are compounds which, by virtue of their particular structure, take over the transfer of chemically bound energy between energy-supplying and energy-consuming processes. Examples of such compounds are glucose-6-phosphate, pyrophosphate, phosphoenol pyruvate, preferably creatine phosphate and more preferably adenosine triphosphate (ATP).

Preparations and/or substances which are capable of increasing the synthesis of energy donors of the mitochondrial respiratory chain and, at the same time, reducing the level of reactive oxygen species (ROS) in the cell metabolism can be, for example, plant extracts or even mixtures of different active substances.

These mixtures preferably contain an antioxidant in combination with at least one other active substance. In a particularly preferred embodiment, they contain vitamin C in combination with yeast and glycogen. It has been found that synergistic effects are produced by the combination of vitamin C, yeast and glycogen.

The active substances are present in the mixtures of antioxidant, yeast and glycogen in a ratio of (1-10):(10-80):(10-80), preferably (3-8):(20-70):(20-60) and more preferably (4-6):(40-60):(30-50).

### DEATILED DESCRIPTION OF THE INVENTION

### Antioxidants

Antioxidants which may be used in the mixtures for stimulating the synthesis of energy donors and for reducing the ROS level are, for example, amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and heptathionine sulfoximine) in very small compatible dosages (for example pmol to μmol/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, superoxide dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids). Of these antioxidants, vitamin C and its derivatives are particularly preferred.

### EXAMPLES

### Method

Human fibroblasts were cultivated for 3 days in a standard medium containing foetal calf serum (FCS). The medium was then replaced by a standard medium containing the active substances to be tested, but no FCS. After incubation for 3 days, cell activity was measured by determining the following parameters.

adenosine triphosphate (ATP)—measured by luminescence based on the enzymatic complex luciferin/luciferase [Vasseur P., Aerts. C., Journal Francais Hydrologie 1981, 9, 149-156]

ROS release from mitochondria—measured by fluorescence using dihydrorhodamine 123 (Rh 123) via the detection of H₂O₂ in the cell cytoplasm (Sakurada, H., Koizumi, H., Ohkawara, A., Ueda, T., Kamo, N., Dermatol. Res., 1992, 284, 144-116].

All values are standardized to the cell protein content by Bradford's method [Bradford, M. M., Anal. Biochem. 1976, 72; 248-254].

### Results

TABLE 1
Level of ATP produced and ROS released in the mitochondrial
respiratory chain after incubation with various compositions of
active substances:

| | | |
|---|---|---|
| | ATP/ | ROS/ |
| Substance used | protein level | protein level |

| | | |
|---|---|---|
| Control | 100% | 100% |
| A - 0.3% by weight [glycogen 50% by | 111% | 199% |
| weight + yeast extract 50% by weight] | | |
| B - 0.3% by weight [glycogen 45% by | 112% | 68% |
| weight + yeast extract 50% by weight + | | |
| vitamin C 5% by weight] | | |
| C - 0.3% by weight [glycogen 40% by | 117% | 212% |
| weight + yeast extract 60% by weight] | | |
| D - 0.3% by weight [glycogen 35% by | 121% | 84% |
| weight + yeast extract 60% by weight + | | |
| vitamin C 5% by weight] | | |

The results show that all the mixtures have an increased ATP level, but only mixtures B and D containing antioxidant also have a reduced ROS level. A synergistic effect is clearly discernible because the addition of 5% by weight of vitamin C itself not only compensates the dramatic increase in ROS to 212% attributable to the increased activity of the mitochondria (increase in energy donor ATP), but actually reduces the quantity of ROS in relation to the standard value without diminishing the positive effect on the concentration of ATP.

The results clearly show that it is possible by selecting suitable active substances to increase the synthesis of energy donors of the mitochondrial respiratory chain but, at the same time, to reduce the level of reactive oxygen species (ROS) in the cell metabolism.

Valuable energy donors are thus made available in the cell metabolism and, in combination with the reduction of cell-damaging ROS, contribute towards preventing and treating aging of the skin and damage to the skin by toxic environmental influences or UV radiation.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

What is claimed is:

1. A process for the production of preparations for treating human skin, characterized in that the preparations contain at least one substance which increases the synthesis of energy donors of the mitochondrial respiratory chain and, at the same time, lowers the level of reactive oxygen species (ROS) in the cell metabolism.

2. The use of cosmetic and/or pharmaceutical preparations containing at least one substance which increases the synthesis of energy donors of the mitochondrial respiratory chain and, at the same time, lowers the level of reactive oxygen species (ROS) in the cell metabolism for treating human skin.

3. The use claimed in claim 2, characterized in that adenosine triphosphate (ATP) and/or creatine phosphate—as energy donors of the mitochondrial respiratory chain—are increased.

4. The use claimed in claim 2, characterized in that the substance is a plant extract.

5. The use claimed in claim 2, characterized in that the substance is an antioxidant in combination with at least one other active substance.

6. The use claimed in claim 2, characterized in that the substance contains vitamin C in combination with yeast and glycogen.

7. A preparation comprising an antioxidant and at least one substance which increases the synthesis of energy donors of the mitochondrial respiratory chain.
